(19) United States
(12) Patent Application Publication
(10) Pub. No.: US 2017/0231810 A1
(43) Pub. Date: Aug. 17, 2017

(54) Therapeutic Wrap Assembly

(71) Applicant: Stephanie Ingram, San Antonio, TX (US)

(72) Inventor: Stephanie Ingram, San Antonio, TX (US)

(21) Appl. No.: 15/043,639

(22) Filed: Feb. 15, 2016

(51) Int. Cl. A61F 7/02

(52) U.S. Cl. CPC A61F 7/02; A61F 2007/0035

(57) ABSTRACT

A therapeutic wrap assembly includes a pouch that may be wrapped around a user's body. The pouch has an outer surface and the outer surface is matable to itself. Thus, the pouch is removably retained on the user's body. A gel is contained within the pouch. The gel may be heated and cooled thereby facilitating the pouch to provide therapeutic heat and therapeutic cooling.

### BACKGROUND OF THE DISCLOSURE

### Field of the Disclosure

The disclosure relates to therapeutic wrap devices and more particularly pertains to a new therapeutic wrap device for providing heat therapy and cold therapy.

### SUMMARY OF THE DISCLOSURE

An embodiment of the disclosure meets the needs presented above by generally comprising a pouch that may be wrapped around a user's body. The pouch has an outer surface and the outer surface is matable to itself. Thus, the pouch is removably retained on the user's body. A gel is contained within the pouch. The gel may be heated and cooled thereby facilitating the pouch to provide therapeutic heat and therapeutic cooling.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a therapeutic wrap assembly according to an embodiment of the disclosure.

FIG. 2 is a top view of an embodiment of the disclosure.

FIG. 3 is a cross sectional view taken along line 3-3 of FIG. 2 of an embodiment of the disclosure.

FIG. 4 is a perspective in-use view of an embodiment of the disclosure.

FIG. 5 is a top perspective view of an embodiment of the disclosure.

### DESCRIPTION OF THE PREFERRED EMBODIMENT

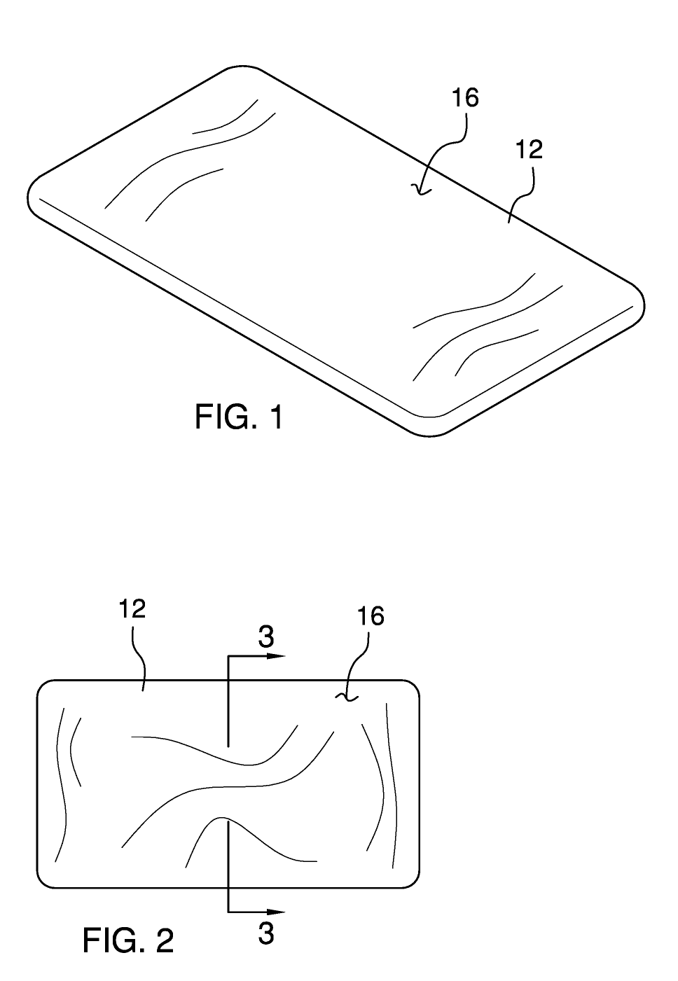

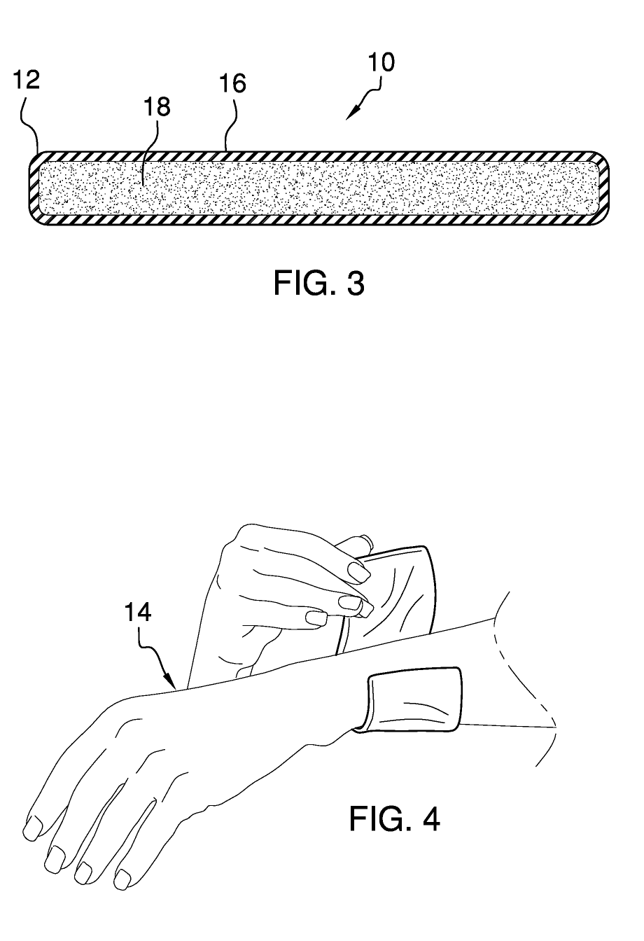

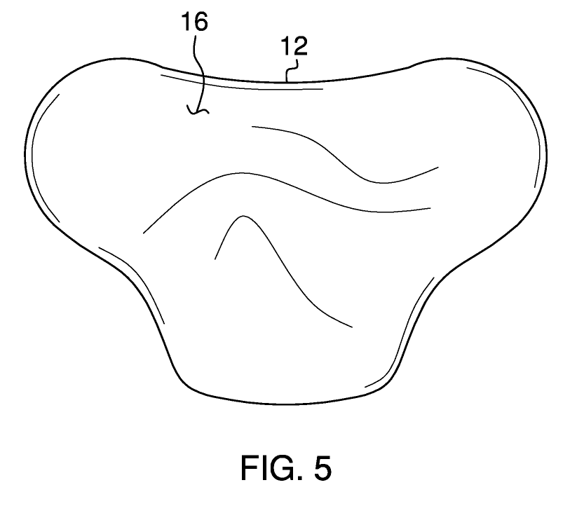

With reference now to the drawings, and in particular to FIGS. 1 through 5 thereof, a new therapeutic wrap device embodying the principles and concepts of an embodiment of the disclosure and generally designated by the reference numeral 10 will be described.

As best illustrated in FIGS. 1 through 5, the therapeutic wrap assembly 10 generally comprises a pouch 12 that may be wrapped around a user's body 14. The pouch 12 has an outer surface 16 and the outer surface 16 is matable to itself. Thus, the pouch 12 may be removably retained on the user's body 14. The pouch 12 may be wrapped around a limb or the like. Additionally, the pouch 12 may be positioned to rest against the user's body 14.

The pouch 12 is comprised of a deformable material. Moreover, the pouch 12 is comprised of a cohesive material. Thus, the pouch 12 sticks to itself and is inhibited from sticking to extrinsic objects. The cohesive material may comprise cohesive silicone or the like.

A gel 18 is contained within the pouch 12. The gel 18 may be heated and cooled. Thus, the pouch 12 may provide therapeutic heat and therapeutic cooling. The gel 18 is comprised of a material having a high thermal mass. The gel 18 may be comprised of non-toxic hot and cold gel or the like. The pouch 12 may come in a variety of shapes, including but not being limited to, a rectangle and triangle.

In use, the pouch 12 is heated or cooled depending upon whether heat therapy is desired or cold therapy is desired. The pouch 12 may be heated in a microwave, an oven or other conventional source of heat. The pouch 12 may be cooled in a refrigerator or the like. Thus, the gel 18 is heated or cooled in the pouch 12. The pouch 12 is wrapped around the user's body 14. The pouch 12 is manipulated to stick to itself. Thus, the pouch 12 is retained on the user's body 14. The pouch 12 may be laid flat on the user's body 14 without having the pouch 12 adhere to itself.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A therapeutic wrap assembly comprising:
a pouch being configured to be wrapped around a user's body, said pouch having an outer surface, said outer surface being matable to itself wherein said pouch is configured to be removably retained on the user's body;
a gel being contained within said pouch, said gel being configured to be heated and cooled thereby facilitating said pouch to provide therapeutic heat and therapeutic cooling.

2. The assembly according to claim 1, wherein said pouch is comprised of a deformable material, said pouch being comprised of a cohesive material such that said pouch sticks to itself and is inhibited from sticking to extrinsic objects.

3. The assembly according to claim 1, wherein said gel is comprised of a material having a high thermal mass.

4. A therapeutic wrap assembly comprising:
a pouch being configured to be wrapped around a user's body, said pouch having an outer surface, said outer surface being matable to itself wherein said pouch is configured to be removably retained on the user's body, said pouch being comprised of a deformable material, said pouch being comprised of a cohesive material such that said pouch sticks to itself and is inhibited from sticking to extrinsic objects; and
a gel being contained within said pouch, said gel being configured to be heated and cooled thereby facilitating said pouch to provide therapeutic heat and therapeutic cooling, said gel being comprised of a material having a high thermal mass.
